(19) United States
(12) Patent Application Publication
(10) Pub. No.: US 2008/0058731 A1
(43) Pub. Date: Mar. 6, 2008

(54) Safety Syringe Device

(76) Inventor: Wei-Shui Wu, Taichung Hsien (TW)

Correspondence Address: HDSL, 4331 STEVENS BATTLE LANE, FAIRFAX, VA, 22033, US

(21) Appl. No.: 11/470,115

(22) Filed: Sep. 5, 2006

(51) Int. Cl. A61M 5/32; A61M 5/315; A61M 5/31; A61M 5/00

(52) U.S. Cl. 604/218; 604/198; 604/240

(57) ABSTRACT

A safety syringe device includes a pusher rod, a connecting sleeve, a syringe tube, a needle base, and a needle cap. The connecting sleeve is sleeved onto the front end of the pusher rod so as to allow insertion of the needle base. After being syringed, the needle base and the acute needle tip is withdrawn together with the pusher rod to be accommodated in the syringe tube, and the emerged main body of the pusher rod is broken so as to insert the needle cover backwardly to the end opening of the syringe tube thereby to avoid exposing the needle tip. The safety syringe device constructed as such is able to avoid hurting any person with the used needle.

### BACKGROUND OF THE INVENTION

1. Field of the Invention

The present invention relates to a safety syringe device, and more particularly to a syringe device, which after completion of syringing, its needle base and used needle can be securely withdrawn to the syringe tube.

2. Description of the Prior Art

Proper disposal of used syringe device especially its acute needle is not only important to the patient and the medical personnel, but also helpful to keep wholesome environment. The improper behaviors, such as repeated use of once for all syringe devoice, especially the needle, or mal-handling the syringe device must be strictly banned. The sanitary regulations in the developed countries ask for strict disposal of used medical instruments and materials such that they should be classified according to the related rules and then packed as a specific package in a tough leak proof, tamper proof, or hermetic container.

One of the safety syringe devices according to the prior arts disclosed in U.S. Pat. No. 60/282,765 is a syringe device applicable for infusing the liquid or powdered drugs into the human body comprising a hollow housing with a proximate and a distal ends used to accommodate aforesaid drugs; a hollow needle inserted to the distal end of the housing and extended longitudinally therein; a plunger with a proximate and a distal end capable of sliding along the proximate end of the housing, and a hollow sleeve slid ably sleeved over the distal end of the housing to cover the needle before syringing, yet retrieved into the housing during syringe operation.

Other disclosures as U.S. Pat. Nos. 5,531,694, 5,632,733, 6,206,857 are all having the safety structures capable of stretching a sleeve from the housing to cover the needle after syringe is completed, or withdrawing the needle into the housing.

For further improving the structures of the safety syringe device described above, the applicant has come out with a newly developed safety syringe device after long time study and experimentation.

### SUMMARY OF THE INVENTION

The present invention is to provide a safety syringe device having a double or singularly threaded end capable of avoiding the needle to be remained on the human skin after completion of syringe.

The present invention is to provide a safety syringe device that the needle base and the needle tip can be withdrawn into the syringe tube after completion of syringe, and particularly the needle base will be securely held in the syringe tube such that avoiding the danger of emerging the needle tip out of the syringe tube.

The present invention is to provide a safety syringe device that the push rod is breakable after completion of syringe so as to shorten the length of the used syringe device convenient for disposal.

The components included in the safety syringe device according to the present invention will be described as follows.

A push rod includes following parts orderly from top to bottom, namely, a top post, a collar, a stem, a stop ring, a stop ring base, a frangible neck, a push rod proper, and an application plate.

A connecting sleeve has a main cavity and a conical cavity to sleeve over the top post, the collar and the stem of the push rod. The connecting sleeve itself is divided into a top, stepped and joint sections, wherein the top section is formed into conical figure, and the joint section is provided with several ribs.

A syringe tube has a tubular space to serve as a container for drugs, the top end thereof is tapered to form a stop portion, and an internal annular protrusion, an open flared portion are successively formed towards the bottom, and finally an application plate is attached externally to the bottom end to be handled with fingers. The stepped section is forwardly tapered to form a hollow conical shape and consists of a retainer ring, several ribs, an upper ring, and a threaded section from top to bottom orderly. The connecting sleeve can be pushed by the push rod to completely cover the stop portion and held at position by the retainer ring and the upper ring.

A needle base has an inner conical tunnel with an acute needle tip formed at the front, the bottom of the needle base is provided with a retainer flange with arcuate sectors.

A hollow needle cover is to protect the needle point.

### BRIEF DESCRIPTION OF THE DRAWINGS

The drawing disclose illustrative embodiments of the present invention which serve to exemplify the various advantages and objects hereof, and are as follows:

FIG. 1 is an exploded view of the present invention;

FIG. 2 is a longitudinal cross sectional view of the present invention;

FIG. 3 is a schematic view of the present invention wherein the connecting sleeve and the push rod are conjoined together;

FIG. 4 is a transverse cross sectional view (1) of the present invention wherein the connecting sleeve and the push rod are conjoined together;

FIG. 5 is transverse cross sectional view (2) of the present invention wherein the connecting sleeve and the push rod are conjoined together;

FIG. 6 is schematic view in an embodiment of the present invention illustrating how to attach the needle base to the syringe tube;

FIG. 7 to FIG. 9 is schematic views in another embodiment of the present invention illustrating how to attach the needle base to the syringe tube;

FIG. 10 is a schematic view of the present invention illustrating how the needle base is tightly combined with the syringe tube by screwing;

FIG. 11 is a transverse cross sectional view of FIG. 10;

FIG. 12 is a transverse cross sectional view of the threaded section of the syringe tube according to the present invention;

FIG. 13 is a transverse cross sectional view of the connecting sleeve according to the present invention;

FIG. 14 is a schematic view of the present invention showing the push rod is completely separated from the needle base; and

FIG. 15 is an illustrative view of the present invention wherein the push rod is pulled out of the syringe tube and the push rod is broken at the frangible neck.

### DETAILED DESCRIPTION OF THE PREFERRED EMBODIMENT

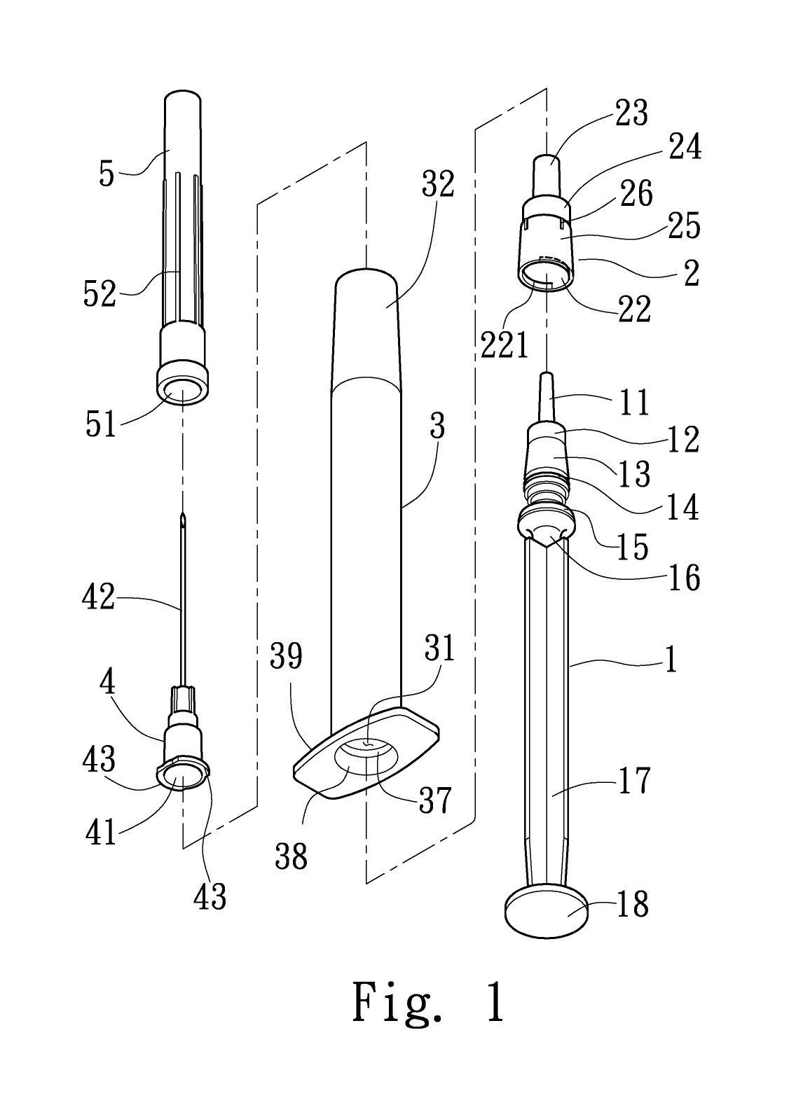

FIG. 1 is an exploded view of the present invention, which shows the safety syringe device, comprises a push rod 1, a connecting sleeve 2, a syringe tube 3, a needle base 4, and a needle cover 5.

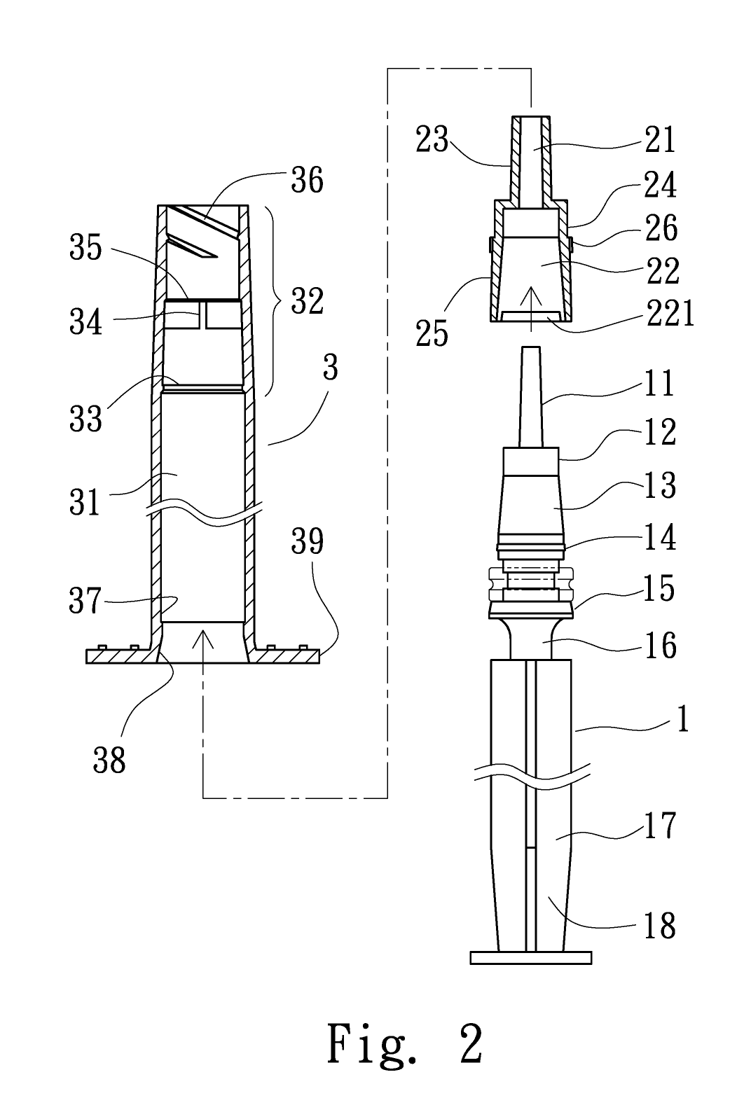

As shown in FIG. 1 and FIG. 2, the push rod is further composed of the following parts orderly from top to bottom, namely a top post 11, a collar 12, a stem 13, a stop ring 14, a stop ring base 15, a frangible neck 16, a push rod proper 17, and an application plate 18. The stop ring 14 and the stop ring base 15 are upwardly tapered.

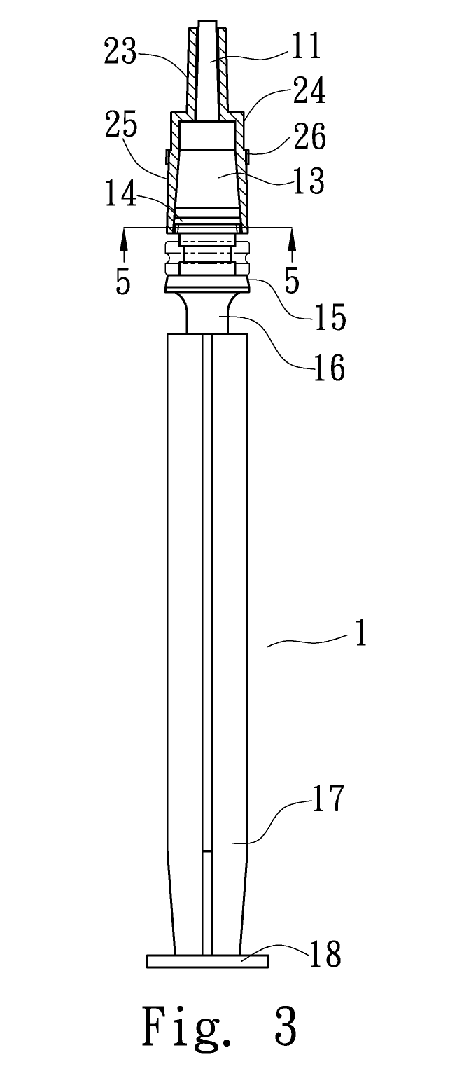

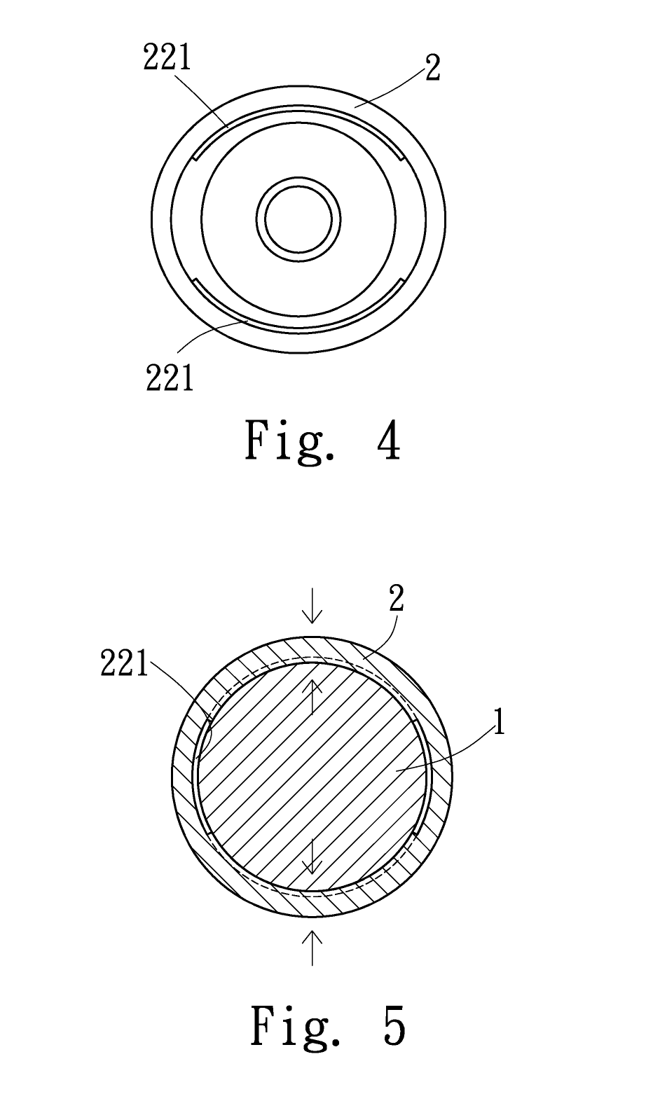

The connecting sleeve 2 having a main cavity 21 and a conical cavity 22 is just to sleeve over the top post 11, the collar 12 and the stem 13 of the push rod 1. As shown in FIG. 3 through FIG. 5, a dimple 221 formed at the bottom of the conical cavity 22 can be coupled with the stop ring 14. The dimple 221 formed at the conical cavity 22 is configured approximately into an ellipse with a total circumferential length shorter than that of the stop ring 14 of the push rod 1 such that the stop ring 14 can be retained tightly in the dimple 221 without the worry of separating accidentally. The connecting sleeve 2 is divided into a top section 23, a stepped middle section 24, and a joint section 25. The top section 23 and the stepped section 24 are tapered, while the joint section 25 is provided with several ribs 26 and at least two adjacent ribs 26 are mutually spaced each other with an angular distance smaller than that of two ribs 34 provided on the syringe tube 3 to be described below.

The syringe tube 3 has a tubular space 31 to serve as a container for drugs, the top end thereof is tapered to form a stop portion 32, and an internal annular protrusion 37, an open flared portion 38 are successively formed towards the bottom, and finally an application plate 39 is attached externally to the bottom end to be handled with fingers. The application plate 39 may be in a circular disc type or the like. The open flared portion 38 is for retaining the stop ring base 15 of the push rod 1 with the stepped annular protrusion 37 so as to prevent the push rod 1 to be tugged out accidentally.

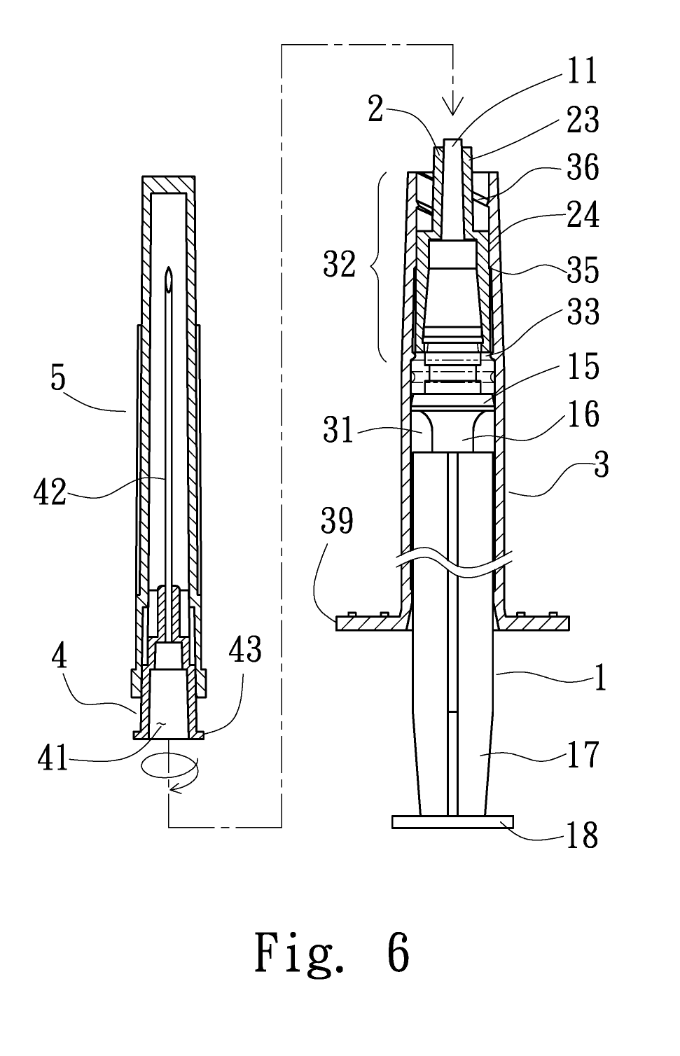

The inner part of the stop portion 32 is forwardly tapered into a hollow conical shape to form, from top to bottom, a retainer ring 33, several ribs 34, an upper ring 35, and a threaded section 36 in order, wherein the ribs 34 are disposed in an equal angular spacing. The threaded section 36 is provided with a singular or a double threading. As shown in FIG. 6, after the connecting sleeve 2 is pushed to reach the stop portion 32 by the push rod 1, it is retained thereat stably by the retainer ring 33 and the upper ring 35 in the manner with its stepped section 24 to tightly bite the inner upper fringe of the upper ring 35 such that a hermetic space is formed between the push rod 1 and the connecting sleeve 2. After that the needle base 4 can be conjoined to the syringe tube 3 by screwing along the threaded section 36. In the conventional safety syringe device, an annular watertight rubber is necessary to be provided around the stepped section 24 of the connecting sleeve 2 so as to keep out moisture. Quite on the contrary, the present invention can do without such a watertight rubber, and instead, by closely urging the inner wall surface of the syringe tube 3 with the conical annular outer surface of the stepped section 24, the aim of water tight can be easily attained that leads to saving the additional component and assembling work.

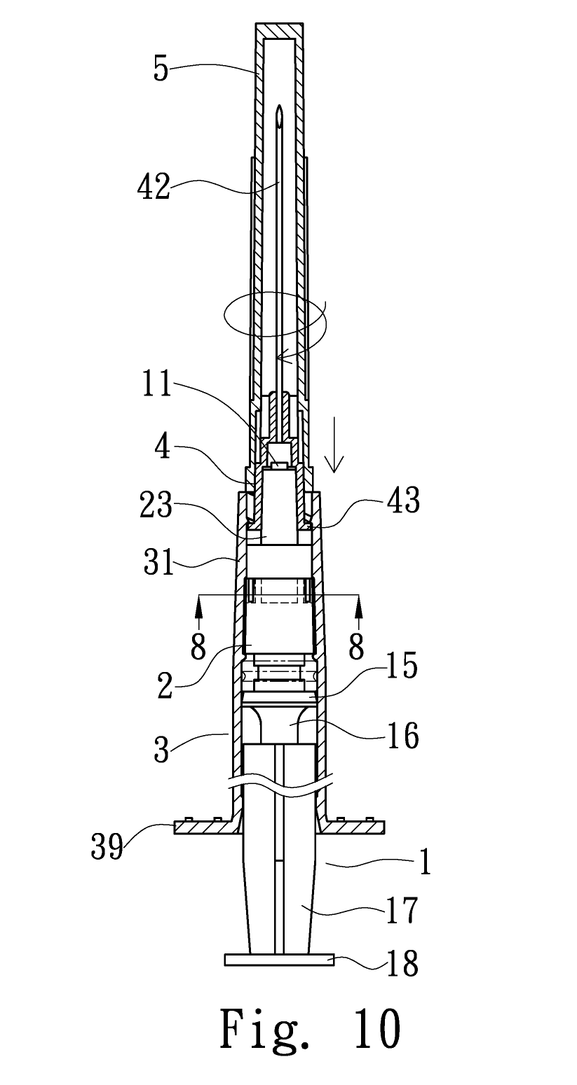

Returning to FIG. 1 and FIG. 6, the needle base 4, consisting of a conical tunnel 41, an upper needle tip 42, and a lower retainer flange 43 can be easily screwed into the syringe tube 3 along the threaded section 36, and then coupled tightly with the top section 23 of the connecting sleeve 2 (see FIG. 10). As the retainer flange 43 is just located under the threaded section 36 and is confined there unable to be released from the threaded section 36, the needle base 4 including the needle tip 42 and the syringe tube 3 is conjoined in one unit. With this structure, to pull out the needle tip 42 from the patient skin after completion of syringe, there is no worry of remaining the needle tip 42 in the patient body by accidental separation of the needle tip 42 from the syringe device.

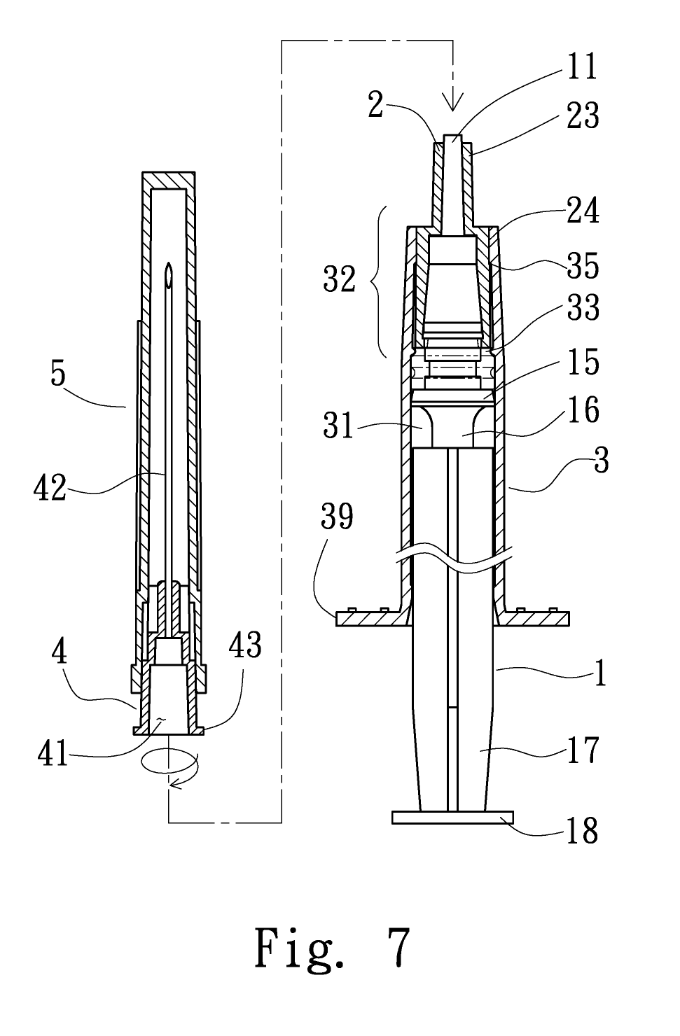

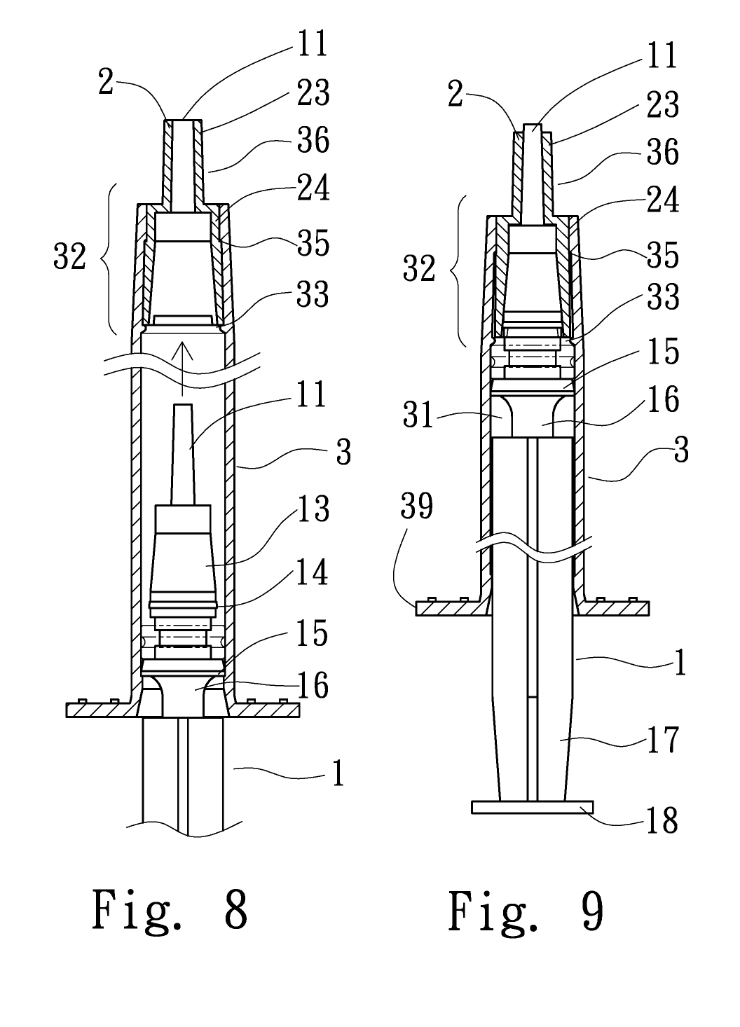

Referring to FIG. 7 through FIG. 9, other than forming the stop portion 32 to a tapered conical hollow body, alternatively, the retainer ring 33, the ribs 34 and the upper ring 35 are replaceable for the function of the threaded section 36. By doing without the threaded section 36, the user may directly push the needle base 4 into the top sleeve section 23 of the connecting sleeve 2 so as to simplify operation.

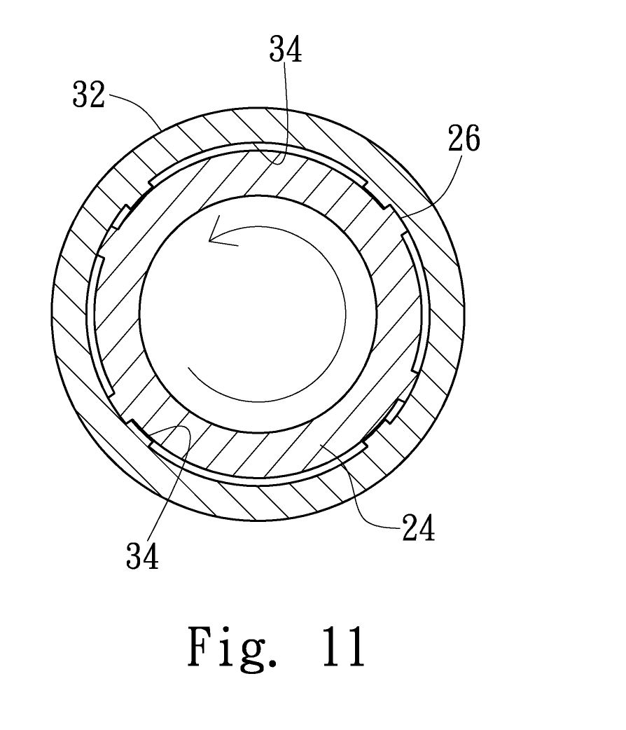

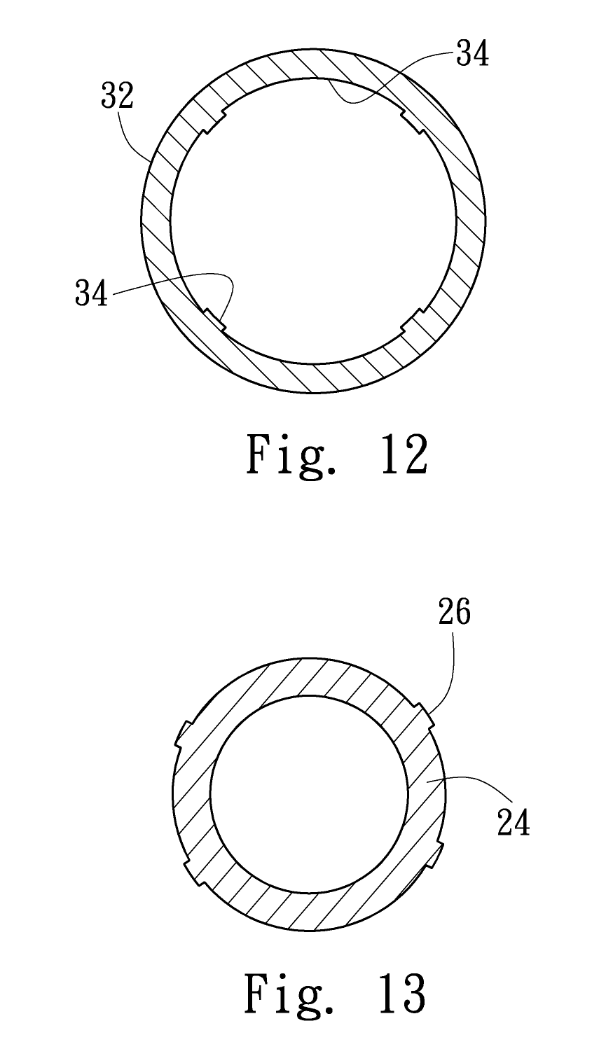

Referring to FIG. 11 through FIG. 13, when the connecting sleeve 2 is pushed to reach the stop portion 32 of the syringe tube 3 by the push rod 1, the ribs 34 thereat and the corresponding ribs 26 collaborate to hold the connecting sleeve 2 at position without slipping so as to facilitate assembling of the needle base 4.

As shown in FIG. 1 through FIG. 6, a needle cover 5 is formed into a hollow conical cavity 51 to protect the needle tip 42 by enclosing it. Several ribs 52 are formed along the external fringe of the needle cover 5. The needle cover 5 is either formed into a through type, or having only one opening.

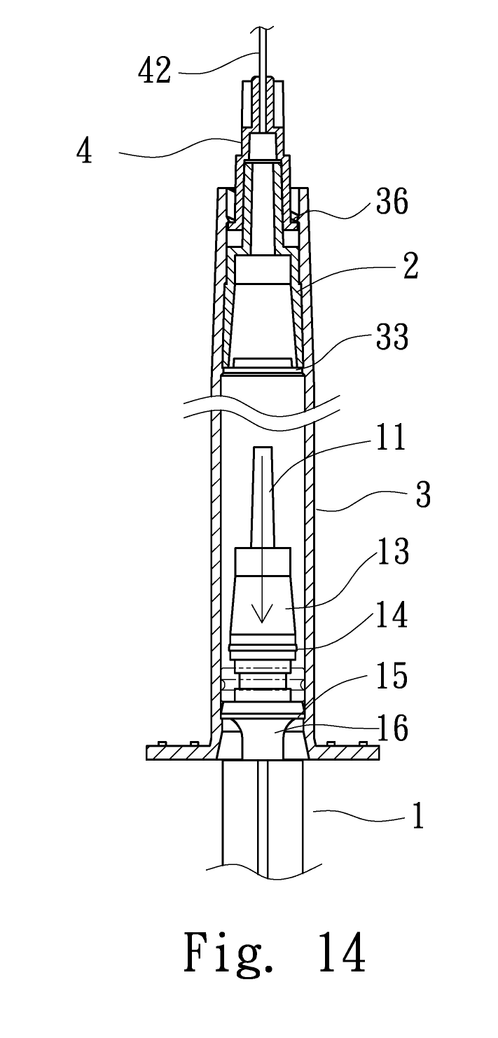

Referring to FIG. 6, FIG. 10 and FIG. 14, After the connecting sleeve 2 is settled at the stop portion 32 of the syringe tube by push rod 1, the needle base 4 along with its needle cover 5 is screwed into the needle tube 3 via threaded section 36 so as to fix the needle base 4 to the top sleeve section 23 of the connecting sleeve 2. After that removing the needle cover 5, and inserting the needle tip 42 into the drugs or patient body, then slowly pulling back the push rod 1 to fill the syringe tube 3 with the drug or draw the blood from the patient's body (see FIG. 14).

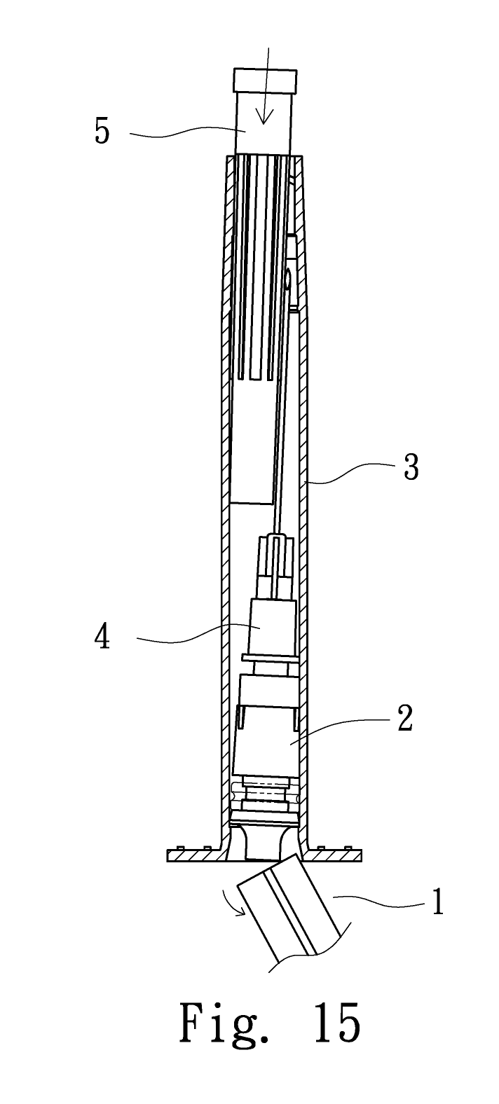

Still referring to FIG. 14, as soon as the syringe is finished, the connecting sleeve 2 is again conjoined with the stem 13 by pushing the push rod 1, at this time the whole needle tip 42 enclosed by the needle cover 5, the push rod 1 is pulled backward and the needle cover 5 is pressed downward to push the whole needle cover 5 including the needle tip 42 into the syringe tube 5. Again pulling out the needle cover 5 and reversely inserting the needle cover 5 into the syringe tube 3 from the stop portion 32, finally breaking the push rod proper 17 of the push rod 1 emerging out of the syringe tube 3 from the frangible neck 16 (see FIG. 15).

Many changes and modifications in the above-described embodiments of the invention can, of course, be carried out without departing from the scope hereof. Accordingly, to promote the progress in science and the useful arts, the invention is disclosed and is intended to be limited only by the scope of the appended claims.

What is claimed is:

1. A safety syringe device comprising:
a push rod which further includes from top to bottom, a top post, a collar, a stem, a top ring, a push rod proper, and an application plate;
a connecting sleeve having a main cavity and a conical cavity to sleeve over the top post, the collar, and the stem of the push rod, wherein the connecting sleeve is divided into a top section, a stepped section, and a joint section, the top section is formed into conical figure, and the joint section is provided with several ribs; and
a syringe tube having a tubular space to serve as a container for drugs, the top end thereof being tapered to form a stop portion, the stop portion being forwardly tapered to form a hollow conical figure, and from bottom to top forming a retainer ring, several ribs and an upper ring, wherein when the connection sleeve is pushed to the stop section by the push rod, the connection sleeve is held at position by the retainer ring and the upper ring.

2. The safety syringe device of claim 1, wherein the safety syringe device further comprises a needle base having a conical cavity a needle tip and a retainer ring beneath the needle tip and has a corresponding arcuate section.

3. The safety syringe device of claim 1, wherein the safety syringe device further comprises a needle covers having a hollow cavity to enclose the needle tip.

4. The safety syringe device of claim 1, wherein the tubular space of the syringe tube has an inner annular protrusion and an open flared portion at a bottom end thereof, and externally attached by an application plate for handling with fingers.

5. The safety syringe device of claim 1, wherein the push rod further comprises a stop ring base and a frangible neck.

6. The safety syringe device of claim 1, wherein the stop ring of the push rod is upwardly tapered.

7. The safety syringe device of claim 5, wherein the stop ring base of the push rod is upwardly tapered.

8. The safety syringe device of claim 1, wherein a dimple is formed at the bottom of the conical cavity of the connecting sleeve, to be coupled with the stop ring of the push rod.

9. The safety syringe device of claim 8, wherein the dimple is configurated into an approximately elliptical figure whose total circumferential length is shorter than that of the stop ring of the push rod.

10. The safety syringe device of claim 1, wherein the syringe tube further comprises a threaded section.

11. The safety syringe device of claim 10, wherein the threads of the threaded section is singular or doubled.

12. The safety syringe device of claim 3, wherein several ribs are provided along external fringe of the needle cover, and the needle cover is a through type or only with one opening.

13. The safety syringe device of claim 1, wherein the angular spacing of adjacent ribs of the connecting sleeve is narrower than that of adjacent ribs of the syringe tube.
